(19) United States
(12) Patent Application Publication
(10) Pub. No.: US 2005/0085807 A1
(43) Pub. Date: Apr. 21, 2005

(54) Instrument with at least two active radio-frequency wires for treatment of tumours

(76) Inventor: Andrea Venturelli, Concesio (IT)

Correspondence Address: SHOEMAKER AND MATTARE, LTD, 10 POST OFFICE ROAD - SUITE 110, SILVER SPRING, MD, 20910, US

(21) Appl. No.: 10/969,208

(22) Filed: Oct. 21, 2004

Related U.S. Application Data
(63) Continuation of application No. PCT/IT03/00250, filed on Apr. 18, 2003.

(30) Foreign Application Priority Data
Apr. 23, 2002 (IT) BS2002U000046

(51) Int. Cl. A61B 18/14

(52) U.S. Cl. 606/41

(57) ABSTRACT

The invention concerns a medical instrument for radio-frequency induced hyperthermia treatment of tumours. It is made up of a guide needle which houses two or more active radio-frequency wires connected at their proximal end to a radio-frequency generator and each one having a spiraled, helicoidal or spiral, distal segment. The wires can be moved longitudinally between a completely retracted passive position in the needle to an active position where the distal segments of the wires protrude from the distal end of the guide needle, automatically adopting a coiled shaped.

### REFERENCE TO EARLIER APPLICATION

This Application is a continuation of International Application No. PCT/IT03/00250, filed Apr. 18, 2003, to Andrea Venturelli, entitled Instrument With At Least Two Active Radio-Frequency Wires For Treatment Of Tumours.

### FIELD OF THE INVENTION

This invention concerns an instrument in the form of a needle-electrode for radio-frequency induced hyperthermia treatment of tumours.

### STATE OF THE ART

Catheter-sounds with at least one terminal electrode or needles with a straight radio-frequency active wire are already available in the treatment of tumoral masses by means of radio frequency induced hyperthermia.

However, the catheter-sounds are relatively large and, even if efficient because of the presence of an important electrode, they have a drawback in that they can only be inserted into the body of the patient until they come into contact with the tumoral mass through natural patent ducts or made patent by dilation.

The needles with an active wire can be introduced into the body by inserting them into the tissues, but their action is limited due to the small diameter of the active wire, which can only influence limited areas and therefore results in longer treatment periods and times. For treatment of larger tumoral masses, larger needles are used, and their use becomes more traumatic for the patient.

In order to improve the efficacy of a radio-frequency active electrode needle in the treatment of very large tumoral masses, keeping nevertheless the diameter within limited dimensions in order to reduce the traumatic effect, a medicinal instrument was projected which includes a hollow guide needle traversed by a single radio-frequency active wire having a terminal segment which is rectilinear when retracted in the needle and becomes helicoidal or spiral when it is in the forward position, emerging from the end of the needle.

In this way, the active wire even if thin is able to irradiate and therefore treat a larger surrounding area in proportion to the area of its cross section.

### OBJECTIVE AND SUMMARY OF THE INVENTION

These radio-frequency active electrode needles are however open to further improvement. One objective of this invention is in fact to supply an electrode needle for hyperthermia treatment of tumours achieved by a new, original combination of the elements having improved functional efficiency and efficacy.

This objective is achieved with a hollow guide needle housing two or more radio-frequency active wires, where said wires can be moved either together or singularly between an inactive retracted position in the guide needle and an active protruding position from a distal end of said needle, and where the wires each have a proximal end which can be connected to a radio frequency generator and a pre-shaped distal part to move from a substantially rectilinear form, when the wire is in the retracted position, to a helicoidal or spiral shape, when the wire is in the active protruding position from the guide needle.

The two or more wires form in this way an equivalent number of electrodes which contemporaneously or selectively activated and/or oriented according to needs enable treatment of vast tumoral areas increasing the efficacy of the instrument and reducing intervention times.

### BRIEF DESCRIPTION OF THE DRAWINGS

Greater details of the invention will become evident from the following description made with reference to the indicative and non-limiting drawings enclosed, in which:

FIG. 1 shows a needle with two wires in the forward position;

FIG. 2 shows an enlarged longitudinal cross-section of a distal part of the needle in FIG. 1 with wires retracted;

FIG. 3 shows a cross-section of the needle in FIG. 1 enlarged even further;

FIG. 4 shows a cross-section of an instrument with wires housed in respective ducts in the guide needle;

FIG. 5 shows an instrument with a wire winding device for moving them in the guide needle; and

FIG. 6 shows a cross section of a variation in construction of the needle.

### DETAILED DESCRIPTION OF THE PREFERRED EMBODIMENTS

As shown, the medical instrument includes a guide needle 11 equipped with a grip 12 and destined to be inserted into the body of the patient until it reaches the tumoral area to be treated.

The guide needle 11 can be made of a tubular element having a longitudinal hole 13 with two or more wires extending from the proximal to the distal part 14, 15 . . . , movable in lengthwise between a fully retracted position of the needle and a forward protruding position of the distal end of the needle itself.

Externally, the guide needle 11 has an insulating sheath 11′ along all its length except for the distal end section 11″.

The wires 14, 15 can each be guided in a separate duct 16, 17, placed inside the guide needle, as shown in FIGS. 2 and 3. Otherwise the wires 15, 15 can be freely housed in the longitudinal hole 13 of the guide needle 11.

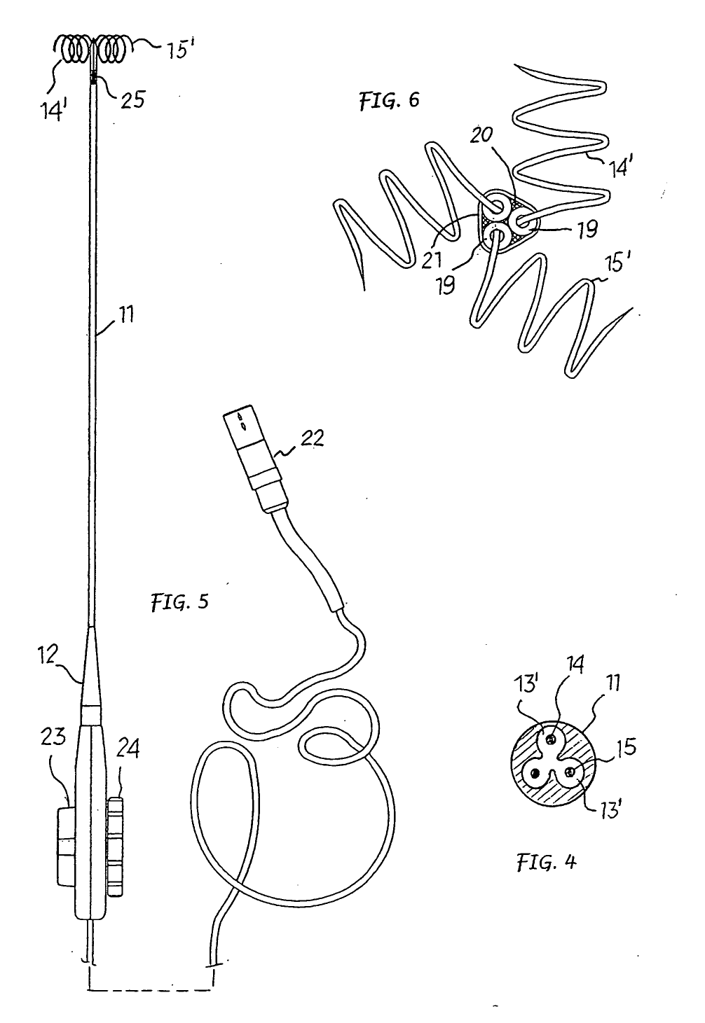

Differently again, as shown in FIG. 4, they can each be housed individually in separate ducts 13′ provided in said hole 13 of the guide needle 11.

In one of the variations, the instrument can have a needle made up of the same number of tubes 19 as the active wires 14, 15 . . . . With a wire in each tube. The tubes 19 are placed side by side and welded together using a resin or something similar 20 and enclosed in an insulating sheath 21, as shown in FIG. 6.

Wires 14, 15 are practically straight except for a distal section 14′, 15′ having respectively, either a helicoidal or spiral configuration, prepared during a pre-shaping process.

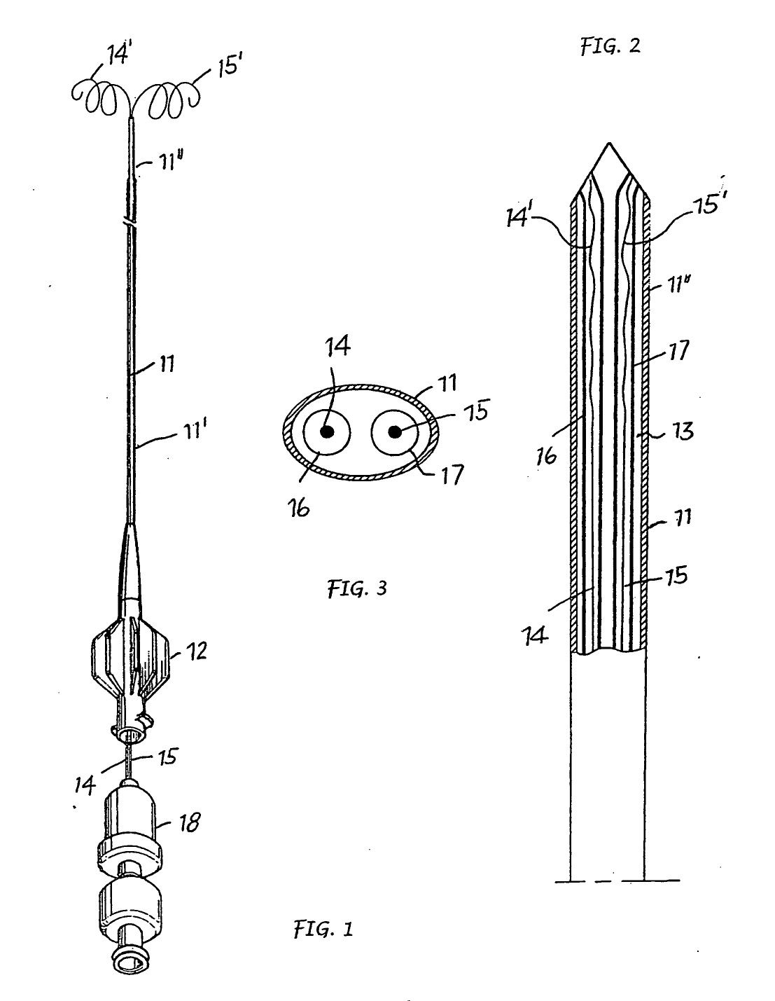

The proximal ends of the wires can all be attached to a connection 18 and connected by means of a connector 22—FIG. 5—to a radio-frequency generator—not shown. In this way wires 14,15 can be moved forwards or backwards at the same time in the needle guide 11 or in the respective tubes 16,17, 19 between a passive position where they are fully retracted inside the needle guide as shown in FIG. 2, and an active position where their terminal segments 14′, 15′ protrude from the distal end of the needle itself having a helicoidal or spiral configuration as shown in FIG. 1 and 5.

As an alternative, each wire 14, 15, . . . can be attached to its own connection and connected, by the latter, to a radio-frequency generator. In this case and according to needs, the wires, each being independent of the other, can be manoeuvred and moved singularly, or together, between the passive and active positions.

Furthermore, the longitudinal movements of the wires between said passive and active positions, may be achieved by means of a winder pulley 23—FIG. 5—to which the proximal ends of the wires are connected and which can be turned using a handle 24 associated with and needle indicating the longitudinal position of the wires.

The helicoidal or spiral segments 14′, 15′ of wires 14, 15 can be oriented in different directions when, protruding from the needle they are in the active position. Orienting the wires can, in addition, be changed by turning the connection or connections the wires, as the case may be, are connected to.

Worthy of note is the fact that the distal end of the guide needle can also be equipped with a thermocouple 25 to read the temperature in the area to be treated with the instrument described herein.

While the needle is being introduced into the body of the patient, the wires 14, 15, . . . are held in the passive position, retracted in the needle. Their distal segments 14′, 15′ . . . remain as it were almost rectilinear, constrained and housed in the needle cavity. When the needle reaches the area to be treated, the distal segments of the wires are made to protrude from the distal end of the needle and automatically become helicoidal or spiral. In this way, once connected to the radio-frequency generator they irradiate the area to be treated, necrotising the ill tissue using hyperthermia induced by radio-frequency.

1. A medical instrument for the treatment of tumors by radio-frequency induced hyperthermia, comprising:
a guide needle having a proximal part with a grip and a distal part, said needle being introduceable into a body by insertion into tissues; and
two or more active radio-frequency wires, which run lengthwise in said guide needle and which are connected at proximal ends thereof to a radio-frequency generator, each of said wires having either a helicoidal or spiral distal segment, said wires being movable longitudinally between a passive position, completely retracted into said needle, and an active position, wherein said distal segments of said wires protrude from said distal part of said guide needle, automatically adopting a coiled shape.

2. A medical instrument according to claim 1, wherein said guide needle is constructed of a tubular element and said wires pass freely in said guide needle.

3. A medical instrument according to claim 1, wherein said guide needle is constructed of a tubular element and said wires are housed longitudinally in grooves provided in said needle.

4. A medical instrument according to claim 1, wherein said guide needle is constructed of a tubular element and said wires are individually housed in corresponding guide tubes placed in said guide needle.

5. A medical instrument according to claim 4, wherein each said wire is turnable within said corresponding guide tube so as to direct said distal segment thereof.

6. A medical instrument according to claim 1, wherein said guide needle is constructed of several small tubes placed side by side, welded together and housed in an insulating sheath, and said wires are individually placed, each one occupying a corresponding one of said small tubes.

7. A medical instrument according to claim 6, wherein each said wire is turnable within said corresponding guide tube so as to direct said distal segment thereof.

8. A medical instrument according to claim 1, wherein said wires, being attached to a proximal connection connected to the radio-frequency generator and a grip for longitudinal movement thereof, can be moved simultaneously between said active and passive positions.

9. A medical instrument according to claim 1, wherein said wires, each one being individually attached to a proximal connection connected to the radio-frequency generator and a grip for longitudinal movement thereof, can be moved individually between said active and passive positions.

10. A medical instrument according to claim 1, wherein said wires, being attached to a proximal wind/unwind device connection, can be moved simultaneously between said active and passive positions.

11. A medical instrument according to claim 1, wherein said wires, being attached individually to a separate proximal wind/unwind device connection, can be moved individually between said active and passive positions.

12. A medical instrument according to claim 1, wherein said guide needle is associated with a thermocouple to read the temperature at said distal part.
